(19) United States
(12) Patent Application Publication
(10) Pub. No.: US 2010/0043819 A1
(43) Pub. Date: Feb. 25, 2010

(54) COMPOSITIONS CONTAINING STIMULUS SENSITIVE COLORANT

(75) Inventors: Sue Feng, Edison, NJ (US); Diane Keene, Burlington, NJ (US); Murat Quadir, Midlothian, VA (US); Kavita Patel, North Brunswick, NJ (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'Oréal S.A., Paris (FR)

(21) Appl. No.: 12/545,329

(22) Filed: Aug. 21, 2009

Related U.S. Application Data
(60) Provisional application No. 61/090,668, filed on Aug. 21, 2008.
(60) Provisional application No. 61/090,696, filed on Aug. 21, 2008.
(60) Provisional application No. 61/090,701, filed on Aug. 21, 2008.

(51) Int. Cl. A45D 2/48; A61K 8/19; A61Q 1/02; A61Q 1/04; A61Q 1/10; A45D 40/26

(52) U.S. Cl. 132/217; 424/63; 424/70.6; 424/70.7; 424/64; 132/218

(57) ABSTRACT

The invention relates to compositions containing stimulus sensitive colorant as well as to methods of applying such compositions to keratin materials and kits containing such compositions.

### CROSS-REFERENCE TO RELATED APPLICATIONS

This application claims the benefit of priority under 35 U.S.C. §119 (e) from U.S. Provisional Application Ser. Nos. 61/090,668, filed Aug. 21, 2008; 61/090,696, filed Aug. 21, 2008; and 61/090,701, filed Aug. 21, 2008, the entire contents of which are hereby incorporated by reference.

### FIELD OF THE INVENTION

The present invention generally relates to compositions comprising stimulus sensitive colorant as well as to methods of applying such compositions to keratin materials and kits comprising such compositions. Such compositions possess improved properties and characteristics such as, for example, allowing the color or shade of a composition to be changed or altered before, during or after application, and/or enhancing the characteristics, appearance or properties of other colorants in the same composition or in a complementary composition.

### BACKGROUND OF THE INVENTION

Cosmetic compositions often contain colorant to provide a coloring effect to the keratin material to which they have been applied. In the past, changing the coloring effect provided by these compositions has been difficult if not impossible. Generally speaking, the only way to change coloring was to apply a different composition having a different colorant over the previously applied composition, or to remove the applied cosmetic composition, replacing it with a different composition containing a different colorant. Obviously, such additional steps and manipulations were time-consuming and/or inconvenient, and inhibited changing the desired coloring effect. Thus, there is a need for improved colorant-containing compositions in which at least some of the colorant components can more easily be changed or altered before, during or after application by the end user.

Accordingly, one aspect of the present invention is a care and/or makeup and/or treatment composition for keratin materials which is able to address or overcome the aforementioned problems with the prior art compositions.

### BRIEF SUMMARY OF THE INVENTION

The present invention relates to compositions comprising at least one stimulus sensitive colorant. Such a composition could be, for example, a topcoat (for example, a mascara, foundation or lipstick) which could be applied to a basecoat (for example, a primer), or a basecoat (for example, a mascara, foundation or lipstick) upon which a topcoat (for example, a gloss or moisturizing composition) could be applied.

For example, the present invention relates to compositions comprising at least one pH or oxidation sensitive colorant. Such a composition could be, for example, a topcoat (for example, a mascara, foundation or lipstick) which could be applied to a basecoat (for example, a primer) comprising at least one pH or oxidation activating agent, or a basecoat (for example, a mascara, foundation or lipstick) upon which a topcoat (for example, a gloss or moisturizing composition) comprising at least one pH or oxidation activating agent could be applied.

The present invention further relates to methods of applying a composition comprising at least one stimulus sensitive colorant to a keratin material.

For example, the present invention further relates to methods of applying a composition comprising at least one pH or oxidation sensitive colorant to a keratin material, before, after or simultaneously with a composition comprising at least one pH or oxidation activating agent.

The present invention also relates to methods of applying to a keratin material a basecoat composition comprising at least one stimulus sensitive colorant, and then applying to the basecoat composition a topcoat composition.

For example, the present invention also relates to methods of applying to a keratin material a basecoat composition comprising at least one pH or oxidation sensitive colorant, and then applying to the basecoat composition a topcoat composition comprising at least one pH or oxidation activating agent.

The present invention also relates to methods of applying to a keratin material a basecoat composition, and then applying to the basecoat composition a topcoat composition comprising at least one stimulus sensitive colorant.

For example, the present invention also relates to methods of applying to a keratin material a basecoat composition comprising at least one pH or oxidation activating agent, and then applying to the basecoat composition a topcoat composition comprising at least one pH or oxidation sensitive colorant.

The present invention also relates to methods of treating, caring for, making up or enhancing the appearance of keratin materials comprising applying compositions of the present invention to the keratin materials in an amount sufficient to treat, care for, make-up and/or enhance the appearance of the keratin materials.

The present invention also relates to kits comprising (a) a basecoat composition comprising at least one stimulus sensitive colorant; and (b) a topcoat composition. The kit can also contain a heating device such as an applicator for subjecting the basecoat composition to pressure and/or heating, if desired.

For example, the present invention also relates to kits comprising (a) a basecoat composition comprising at least one pH or oxidation sensitive colorant; and (b) a topcoat composition comprising at least one pH or oxidation activating agent.

The present invention also relates to kits comprising (a) a basecoat composition; and (b) a topcoat composition comprising at least one stimulus sensitive colorant. The kit can also contain a heating device such as an applicator for subjecting the topcoat composition to pressure and/or heating, if desired.

For example, the present invention also relates to kits comprising (a) a basecoat composition comprising at least one pH or oxidation activating agent; and (b) a topcoat composition comprising at least one pH or oxidation sensitive colorant.

The present invention further relates to methods of changing the color or appearance of a cosmetic composition comprising at least one stimulus sensitive colorant comprising applying a stimulus (for example, pressure change, temperature change such as heating or cooling, pH or oxidation change) to the composition to effect a change in color or appearance of the composition. Such application of stimulus could occur before, during or after application of the cosmetic composition to a keratin material.

For example, the present invention further relates to methods of changing the color or appearance of a cosmetic composition comprising at least one pH or oxidation sensitive colorant comprising applying to the composition a second composition comprising at least one pH or oxidation activating agent to effect a change in color or appearance of the composition.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

“Cosmetically acceptable” means that the item in question is compatible with any keratin material. For example, “cosmetically acceptable medium” means a medium that is compatible with any keratin material.

“Keratin material” includes, for example, skin, hair, nails, eyelashes, eyelids, eyebrows, lips and any other area of body or facial skin.

The compositions, methods and kits of the present invention can comprise, consist of, or consist essentially of the essential elements and limitations of the invention described herein, as well as any additional or optional ingredients, components, or limitations described herein or any otherwise useful ingredient found in personal care compositions intended for application to keratin materials.

The composition of the present invention may be in any form suitable for use on keratin materials such as, for example, solid, liquid, anhydrous, oil-free or emulsion compositions (for example, water-in-oil emulsion, oil-in-water emulsion, multiple emulsion (W/O/W or O/W/WO), suspensions, dispersions, nanoemulsions, etc.). The compositions of the present invention can be, for example, temporary tattoos, foundations, eyeshadows, lipsticks, liquid lip compositions, nail polishes, mascaras, eyeliners, hair dyes, etc. The compositions of the present invention can be used as a basecoat and/or topcoat for application beneath and/or onto other products applied to keratin materials.

As defined herein, stability is tested by placing the composition in a controlled environment chamber for 8 weeks at 25° C. In this test, the physical condition of the sample is inspected as it is placed in the chamber. The sample is then inspected again at 24 hours, 3 days, 1 week, 2 weeks, 4 weeks and 8 weeks. At each inspection, the sample is examined for abnormalities in the composition such as phase separation if the composition is in the form of an emulsion. The stability is further tested by repeating the 8-week test at 4° C., 37° C., 45° C. and/or under freeze-thaw conditions. A composition is considered to lack stability if in any of these tests an abnormality that impedes functioning of the composition is observed. The skilled artisan will readily recognize an abnormality that impedes functioning of a composition based on the intended application.

Stimulus Sensitive Colorant

According to the present invention, compositions comprising at least one stimulus sensitive colorant are provided. In accordance with the present invention, any suitable, cosmetically acceptable pressure sensitive colorant, temperature sensitive colorant, pH or oxidation sensitive colorant, or any combination of these colorants, can be used as stimulus sensitive colorant.

Temperature Sensitive Colorant (Temperature Activated Colorants)

According to preferred embodiments of the present invention, compositions comprising at least one temperature sensitive colorant are provided. In accordance with these preferred embodiments, any suitable, cosmetically acceptable temperature sensitive colorant can be used. “Temperature sensitive colorant” as used herein means any colorant which visibly changes color upon a change in temperature applied to the composition (either by heating or cooling). For example, the temperature applied to the composition can be applied by prolonged direct exposure to sunlight (for example, a sunscreen composition comprising at least one temperature sensitive colorant can change color after sufficient, direct exposure to sunlight, thereby indicating that sunscreen should be reapplied or that exposure to sun should be terminated), by applicator (for example, a mascara or nail polish applicator could be used to heat mascara or nail polish during application), by friction (for example, a foundation could be rubbed with fingers before, during or after application to heat the foundation), or by external device (for example, a nail polish or liquid lip composition could be heated by a heat lamp or cooled in front of an air conditioner after application to change color). Preferably, the visual change in color occurs after a change of at least 25° C., more preferably at least 28° C. Suitable examples of acceptable temperature sensitive colorants include, but are not limited to, those inks disclosed in U.S. Pat. No. 6,498,203, the entire contents of which is hereby incorporated by reference in its entirety, as well as DYNACOLOR THERMOCHROMIC SLURRY POWDER COLD COLOR: BLUE; WARM COLOR: COLORLESS—CHROMATIC TECHNOLOGIES INC—activating temperature 53° C.), DYNACOLOR THERMOCHROMIC WET SLURRY (COLD COLOR: BLUE; WARM COLOR: COLORLESS—CHROMATIC TECHNOLOGIES INC—activating temperature 53° C.), DYNACOLOR THERMOCHROMIC WET SLURRY (COLD COLOR: BLACK; WARM COLOR: COLORLESS—CHROMATIC TECHNOLOGIES INC—activating temperature 55° C.), and DYNACOLOR THERMOCHROMIC SLURRY POWDER (COLD COLOR: BLACK; WARM COLOR: COLORLESS—CHROMATIC TECHNOLOGIES INC—activating temperature 60° C.).

According to one embodiment of the present invention, compositions comprising at least one temperature sensitive colorant are free of (that is, contain less than 0.25% by weight) or substantially free of (that is, contain less than 1.0% by weight) other non temperature sensitive colorants.

Preferably, the temperature sensitive colorant(s) is present in the composition in an amount ranging from about 0.1% to about 50% by weight with respect to the total weight of the composition, more preferably from about 0.5% to about 40% by weight with respect to the total weight of the composition, more preferably from about 1% to about 30% by weight with respect to the total weight of the composition, and more preferably from about 1% to about 25% by weight with respect to the total weight of the composition, including all ranges and subranges therebetween.

pH Or Oxidation Sensitive Colorant (pH Or Oxidation Activated Colorant)

According to preferred embodiments of the present invention, compositions comprising at least one pH or oxidation sensitive colorant are provided. In accordance with these preferred embodiments, any suitable, cosmetically acceptable pH or oxidation sensitive colorant can be used. “pH or oxidation sensitive colorant” as used herein means any colorant which visibly changes color upon contact with a “pH or oxidation activating agent.”

For example, suitable pH or oxidation sensitive colorants can include Lewis acids, and suitable pH or oxidation activating agents can include Lewis bases. Alternatively, suitable pH or oxidation sensitive colorants can include Lewis bases, and suitable pH or oxidation activating agents can include Lewis acids.

Specific examples of acceptable pH or oxidation activating agents include, but are not limited to, Lewis acids such as citric acid, bis-phenol A and green tea extract (epicatechin).

Examples of acceptable pH or oxidation sensitive colorants include, but are not limited to, leuco dye intermediates which are sensitive to oxidation and/or acidic environments. Examples of suitable leuco dyes include, but are not limited to, diarylphthalide dyes, fluoran dyes, indolyphthalide dyes, acylluecoazine dyes, leucoauramine dyes, spiropyrane dyes, rhodaminelactam dyes, triarylmethane dyes and chromene dyes. Other examples include, but are not limited to, diarylmethane derivative dyes, triarylmethane derivative dyes, and methine derivative dyes.

Specific examples of suitable dyes include, but are not limited to, Hodogaya Green DCF, Hodogaya Orange DCF, Hodogaya Blue NC-1, Copikem #4 Black, Hodogaya NC-Black-1, Copikem #37 Yellow, Ciba Pergascript Yellow, T Spiro(12H-benzo(a)xanthene-12,1′(3′H)-isobenzofuran-3′-one,9-(diethylamino) (such as COPIKEM®747), 3-[Butyl-2-methylindol-3-yl]-3-(1-octyl-2-methylindol-3-yl)-1(3H)isobenzofuranone (such as COPIKEM®35 magenta), 2-′phenylamino-3′-methyl-6′-(dibutylamino) spiro[isobenzofuran-1(3H), 9′-(9H)-xanthen]-3-one (such as COPIKEM®34 Black), substituted Phthalide (such as COPIKEM®14 Orange), such as COPIKEM®7 Grape, 2′Di(phenylmethyl)amino-6′(diethylamino)spiro(isobenzofuran-1(3H), 9′-(9H)xanthen)-3-one (such as COPIKEM®5 green). Products identified under the COPIKEM® trademarks are commercially available from the Hilton Davis Company, Cincinnati, Ohio.

Further dyes are disclosed in U.S. Pat. Nos. 7,229,487 and 6,124,377, the entire contents of both of which are hereby incorporated by reference.

According to one embodiment of the present invention, compositions comprising at least one pH or oxidation sensitive colorant are free of (that is, contain less than 0.25% by weight) or substantially free of (that is, contain less than 1.0% by weight) other colorants.

Preferably, the pH or oxidation sensitive colorant(s) is present in the composition in an amount ranging from about 0.1% to about 50% by weight with respect to the total weight of the composition, more preferably from about 0.5% to about 40% by weight with respect to the total weight of the composition, more preferably from about 1% to about 30% by weight with respect to the total weight of the composition, and more preferably from about 1% to about 25% by weight with respect to the total weight of the composition, including all ranges and subranges therebetween.

pH Or Oxidation Activating Agents

According to preferred embodiments of the present invention, compositions comprising at least one pH or oxidation activating agent are provided. Suitable activating agents are discussed above. In accordance with these preferred embodiments, the composition comprising the pH or oxidation sensitive colorant and the composition comprising the pH or oxidation activating agent are different compositions. That is, the pH or oxidation sensitive colorant and the pH or oxidation activating agent are not in the same composition.

Preferably, the pH or oxidation activating agent(s) is present in the composition in an amount ranging from about 0.1% to about 50% by weight with respect to the total weight of the composition, more preferably from about 0.5% to about 40% by weight with respect to the total weight of the composition, more preferably from about 1% to about 30% by weight with respect to the total weight of the composition, and more preferably from about 1% to about 25% by weight with respect to the total weight of the composition, including all ranges and subranges therebetween.

Pressure Sensitive Colorant (Pressure Activated Colorant)

According to preferred embodiments of the present invention, compositions comprising at least one pressure sensitive colorant are provided. In accordance with these embodiments, any suitable, cosmetically acceptable pressure sensitive colorant can be used. “Pressure sensitive colorant” as used herein means any colorant which visibly changes color upon application of pressure to the composition. For example, the pressure applied to the composition can be applied by applicator (for example, the nozzle of a mascara or nail polish applicator could be used to apply pressure to the mascara or nail polish during application), by hand (for example, a foundation could be rubbed with fingers before, during or after application while applying pressure), or by external device (for example, a nail polish or liquid lip composition could be subjected to pressure after application using a device such as, for example, a pad, to change color, or an eyelash crimping device could be used to change color of at least a portion of mascara which has been applied to eyelashes).

According to one embodiment of the present invention, compositions comprising at least one pressure sensitive colorant are free of (that is, contain less than 0.25% by weight) or substantially free of (that is, contain less than 1.0% by weight) other colorants.

Preferably, the pressure sensitive colorant(s) is present in the composition in an amount ranging from about 0.1% to about 50% by weight with respect to the total weight of the composition, more preferably from about 0.5% to about 40% by weight with respect to the total weight of the composition, more preferably from about 1% to about 30% by weight with respect to the total weight of the composition, and more preferably from about 1% to about 25% by weight with respect to the total weight of the composition, including all ranges and subranges therebetween.

According to preferred embodiments of the present invention, the pressure sensitive colorant is an encapsulated colorant. In accordance with these embodiments, applying pressure to the composition results in destruction of the encapsulation, thereby releasing colorant from the encapsulation. If the colorant possesses different color characteristics than the encapsulating material, release of the colorant from encapsulation may change the coloration of the composition.

Also, if the colorant is a pH or oxidation sensitive colorant and (1) the composition further comprises a pH or oxidation activating agent or (2) the composition is used in conjunction with a second composition comprising a pH or oxidation activating agent, release of the colorant from encapsulation so that it can be activated by the pH or oxidation activating agent can change the coloration of the composition. Thus, according to particularly preferred embodiments, compositions of the present invention comprise at least one pH or oxidation activating agent and at least one pressure sensitive colorant which is an encapsulated pH or oxidation sensitive colorant.

In accordance with these preferred embodiments, any suitable, cosmetically acceptable pH or oxidation sensitive colorant can be used. “pH or oxidation sensitive colorant” as used herein means any colorant which visibly changes color upon contact with a “pH or oxidation activating agent.”

For example, suitable pH or oxidation sensitive colorants can include Lewis acids, and suitable pH or oxidation activating agents can include Lewis bases. Alternatively, suitable pH or oxidation sensitive colorants can include Lewis bases, and suitable pH or oxidation activating agents can include Lewis acids.

Specific examples of acceptable pH or oxidation activating agents include, but are not limited to, Lewis acids such as citric acid, bis-phenol A and green tea extract (epicatechin).

Examples of acceptable pH or oxidation sensitive colorants include, but are not limited to, leuco dye intermediates which are sensitive to oxidation and/or acidic environments. Examples of suitable leuco dyes include, but are not limited to, diarylphthalide dyes, fluoran dyes, indolyphthalide dyes, acylluecoazine dyes, leucoauramine dyes, spiropyrane dyes, rhodaminelactam dyes, triarylmethane dyes and chromene dyes. Other examples include, but are not limited to, diarylmethane derivative dyes, triarylmethane derivative dyes, and methine derivative dyes.

Specific examples of suitable dyes include, but are not limited to, Hodogaya Green DCF, Hodogaya Orange DCF, Hodogaya Blue NC-1, Copikem #4 Black, Hodogaya NC-Black-1, Copikem #37 Yellow, Ciba Pergascript Yellow, T Spiro(12H-benzo(a)xanthene-12,1′(3′H)-isobenzofuran-3′-one,9-(diethylamino) (such as COPIKEM®747), 3-[Butyl-2-methylindol-3-yl]-3-(1-octyl-2-methylindol-3-yl)-1(3H)isobenzofuranone (such as COPIKEM®35 magenta), 2-′phenylamino-3′-methyl-6′-(dibutylamino) spiro[isobenzofuran-1(3H),9′-(9H)-xanthen]-3-one (such as COPIKEM®34 Black), substituted Phthalide (such as COPIKEM®14 Orange), such as COPIKEM®7 Grape, 2′Di(phenylmethyl)amino-6′(diethylamino)spiro(isobenzofuran-1(3H), 9′-(9H)xanthen)-3-one (such as COPIKEM®5 green). Products identified under the COPIKEM® trademarks are commercially available from the Hilton Davis Company, Cincinnati, Ohio.

Further dyes are disclosed in U.S. Pat. Nos. 7,229,487 and 6,124,377, the entire contents of both of which are hereby incorporated by reference.

Also in accordance with preferred embodiments of the present invention, any suitable encapsulating material can be used. Suitable examples of such materials include, but are not limited to, acrylic, polyamide, polyurethane, vinyl, acrylamide, urea-formaldehyde, epoxy, phenolics, polystyrene, polyurea, rosin, starch, gluten, cellulosic, gelatin, or combinations thereof. It will be appreciated that the microcapsules may be produced by a variety of materials known in the art, and that one of skill in the art would be able to select the proper shell material for encapsulation purposes.

For example, U.S. patent application publication no. 2007/0244219 and U.S. Pat. Nos. 4,087,376, 4,001,140, 4,273,672, 5,961,804, 6,375,872, and 6,592,990. the entire contents of all of which are hereby incorporated by reference, disclose the manufacture of encapsulated materials.

Particularly preferred encapsulated colorants include microencapsulated leuco dye ink sold by Microtek.

Further in accordance with preferred embodiments, compositions comprising at least one pH or oxidation activating agent are provided. Suitable activating agents are discussed above. Preferably, the pH or oxidation activating agent(s) is present in the composition in an amount ranging from about 0.1% to about 50% by weight with respect to the total weight of the composition, more preferably from about 0.5% to about 40% by weight with respect to the total weight of the composition, more preferably from about 1% to about 30% by weight with respect to the total weight of the composition, and more preferably from about 1% to about 25% by weight with respect to the total weight of the composition, including all ranges and subranges therebetween.

According to such embodiments, the pH or oxidation activating agent is present in an amount sufficient to activate the pH or oxidation sensitive colorant, thereby changing its color and the color of the composition. Preferably, the molar ratio of pH or oxidation sensitive colorant(s) to pH or oxidation activating agent(s) present in the composition ranges from about 10:1 to about 1:10, more preferably from about 5:1 to about 1:5, and most preferably from about 3:1 to about 1:3, including all ranges and subranges therebetween.

Additional Ingredients

The compositions of the present invention can also comprise any additive usually used in cosmetic or dermatologic compositions. Of course, the particular ingredients added will depend upon the type of composition being prepared. Suitable examples of additional ingredients include, but are not limited to, waxes, film forming agents, dispersants, antioxidants, oils, preserving agents, fragrances, fillers, neutralizing agents, surfactants, cosmetic and dermatological active agents such as, for example, emollients, moisturizers, vitamins, anti-wrinkle agents, essential fatty acids, and sunscreens, and mixtures thereof can be added. A non-exhaustive listing of such ingredients can be found in U.S. patent application Ser. No. 10/733,467, filed Dec. 12, 2003, the entire contents of which is hereby incorporated by reference. Further examples of suitable additional components can be found in the other references which have been incorporated by reference in this application. Still further examples of such additional ingredients may be found in the International Cosmetic Ingredient Dictionary and Handbook (9^{th} ed. 2002).

A person skilled in the art will take care to select the optional additional additives and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

These additives may be present in the composition in a proportion from 0% to 99% (such as from 0.01% to 90%) relative to the total weight of the composition and further such as from 0.1% to 50% (if present), including all ranges and subranges therebetween.

Needless to say, the composition of the invention should be cosmetically or dermatologically acceptable, i.e., it should contain a non-toxic physiologically acceptable medium and should be able to be applied to the eyelashes of human beings.

Specific examples of additional ingredients include oils, particularly if the composition is an anhydrous composition or an emulsion. Any oils can be used in accordance with the present invention. The oils can be volatile or non-volatile, silicone-based and/or hydrocarbon-based, etc. Thus, for example, the external oil phase may contain, independently or in combination, volatile silicone oils, non-volatile silicone oils, volatile non-silicone oils and non-volatile non-silicone oils.

In one embodiment, the compositions of the present invention are substantially free of silicone oils (i.e., contain less than about 1% of silicone oil). In another embodiment, the compositions are substantially free of non-silicone oils (i.e., contain less than about 1% of non-silicone oil). In another embodiment, the compositions are substantially free of non-volatile oils (i.e., contain less than about 1% of non-volatile oil). In yet another embodiment, the compositions are substantially free of volatile oils (i.e., contain less than about 1% of volatile oil).

Water, when present, preferably represents from about 1% to about 70% by weight of the total weight of the composition, more preferably from about 5% to about 60% of the total weight of the composition, and most preferably from about 10% to about 50%, including all ranges and subranges therebetween.

The compositions may also optionally comprise at least one non-pressure sensitive coloring agent. Suitable non-pressure sensitive coloring agents include but are not limited to pigments, dyes, such as liposoluble dyes, and nacreous pigments.

Representative liposoluble dyes which may be used according to the present invention include Sudan Red, DC Red 17, DC Green 6, β-carotene, soybean oil, Sudan Brown, DC Yellow 11, DC Violet 2, DC Orange 5, annatto, and quinoline yellow. The liposoluble dyes, when present, generally have a concentration ranging up to 20% by weight of the total weight of the composition, such as from 0.0001% to 6%, including all ranges and subranges therebetween.

The nacreous pigments which may be used according to the present invention may be chosen from white nacreous pigments such as mica coated with titanium or with bismuth oxychloride, colored nacreous pigments such as titanium mica with iron oxides, titanium mica with ferric blue or chromium oxide, titanium mica with an organic pigment chosen from those mentioned above, and nacreous pigments based on bismuth oxychloride. The nacreous pigments, if present, may be present in the composition in a concentration ranging up to 50% by weight of the total weight of the composition, such as from 0.1% to 20%, preferably from 0.1% to 15%, including all ranges and subranges therebetween.

The pigments, which may be used according to the present invention, may be chosen from white, colored, inorganic, organic, polymeric, nonpolymeric, coated and uncoated pigments. Representative examples of mineral pigments include titanium dioxide, optionally surface-treated, zirconium oxide, zinc oxide, cerium oxide, iron oxides, chromium oxides, manganese violet, ultramarine blue, chromium hydrate, and ferric blue. Representative examples of organic pigments include carbon black, pigments of D & C type, and lakes based on cochineal carmine, barium, strontium, calcium, and aluminum.

If present, the pigments may be present in the composition in a concentration ranging up to 50% by weight of the total weight of the composition, such as from 0.5% to 40%, and further such as from 2% to 30%, including all ranges and subranges therebetween. In the case of certain products, the pigments, including nacreous pigments, may, for example, represent up to 50% by weight of the composition.

According to preferred embodiments of the present invention, methods of applying a composition comprising at least one stimulus sensitive colorant are provided.

According to embodiments including a pH or oxidation sensitive colorant, a first composition comprising at least one pH or oxidation sensitive colorant and a second composition comprising at least one pH or oxidation activating agent are provided. Preferably, the pH or oxidation activating agent is present in an amount sufficient to activate the pH or oxidation sensitive colorant, thereby changing its color. Preferably, the molar ratio of pH sensitive colorant(s) present in one composition to pH or oxidation activating agent(s) present in the other composition ranges from about 10:1 to about 1:10, more preferably from about 5:1 to about 1:5, and most preferably from about 3:1 to about 1:3, including all ranges and subranges therebetween.

According to preferred embodiments, the first and second compositions are combined prior to application to the keratin material to change the color of the pH or oxidation sensitive coloring agent prior to application to the keratin material.

According to other preferred embodiments, methods of applying a composition comprising at least one stimulus sensitive colorant to a keratin material comprising applying to the keratin material a basecoat composition comprising at least one stimulus sensitive colorant, and then applying to the basecoat composition a topcoat composition are provided. For example, a liquid lip composition comprising at least one stimulus sensitive colorant is applied to lips, and then a topcoat composition comprising oils and/or emollients is applied to the basecoat composition. According to preferred embodiments, the basecoat comprises an encapsulated pH or oxidation sensitive colorant and the topcoat comprises a pH or oxidation activating agent.

According to other preferred embodiments, methods of applying a composition comprising at least one stimulus sensitive colorant to a keratin material comprising applying to the keratin material a basecoat composition, and then applying to the basecoat composition a topcoat composition comprising at least one stimulus sensitive colorant are provided. For example, a basecoat primer is applied to eyelashes, lips or skin, and then a topcoat composition comprising at least one stimulus sensitive colorant is applied to the basecoat. Preferably, such a primer basecoat facilitates adhesion of the topcoat comprising at least one stimulus sensitive colorant to the keratin material. According to preferred embodiments, the basecoat comprises a pH or oxidation activating agent and the topcoat comprises an encapsulated pH or oxidation sensitive colorant.

According to particularly preferred embodiments of the present invention, the composition(s) are applied in such a way as to create decorative patterns on the keratin material. For example, the composition comprising at least one stimulus sensitive colorant can be applied in a pattern (for example, stripes, polka dots, etc.) on the keratin material. Such patterns, when initially applied to the keratin materials, could be invisible owing to the coloration of the colorant itself, and/or to the coloration of other pigments present in the composition itself and/or in the complimentary topcoat or basecoat compositions. However, after application of appropriate stimulus, the patterns could emerge. Such methods could be desirable in situations where it might be desirable to keep such patterns hidden (for example, at work) but where it might be desirable to make such patterns visible at a later time (for example, going out after work). Such methods would allow for quick, decorative coloration effects to be visible with simple, quick application of stimulus.

According to other embodiments, after application of the composition to a keratin material, a stimulus could be applied to specific areas of the applied composition, thereby resulting in the formation of the desired pattern. For example, a conventional eyelash curling device could be used to heat or apply pressure to eyelashes at intervals along the eyelashes to create a striped effect. Alternatively, for example, a first composition comprising at least one pH or oxidation sensitive colorant could be applied over a keratin material, and then a second composition comprising at least one pH or oxidation activating agent could be applied in a desired pattern over the first composition.

According to still other preferred embodiments of the present invention, methods of treating, caring for, making up or enhancing the appearance of keratin materials comprising applying compositions of the present invention to the keratin materials in an amount sufficient to treat, care for, make-up and/or enhance the appearance of the keratin materials are provided.

In accordance with all of the foregoing embodiments of the present invention, the compositions of the present invention may be applied to keratin materials as needed, preferably once or twice daily, more preferably once daily and then preferably allowed to dry before subjecting to contact such as with clothing or other objects.

According to yet other embodiments of the present invention, methods of changing the color or appearance of a cosmetic composition comprising at least one stimulus sensitive colorant comprising subjecting the composition to stimulus to effect a change in color or appearance of the composition are provided. As indicated above, such application of stimulus could occur before, during or after application of the cosmetic composition to a keratin material, depending upon the source of stimulus as well as the desired coloring effect (for example, whether an immediate or delayed color change is desired).

The present invention also envisages kits and/or prepackaged materials suitable for consumer use containing one or more compositions according to the description herein (for example, kits containing (1) a composition for application to keratin material such as mascara, eyeliner, a lip composition such as a lipstick or liquid lip color, foundation, etc.; and (2) a basecoat and/or topcoat). According to particularly preferred embodiments, kits comprising (a) a basecoat composition comprising at least one stimulus sensitive colorant, preferably an encapsulated pH or oxidation sensitive colorant; and (b) a topcoat composition, preferably comprising at least one pH or oxidation activating agent, are provided. According to other particularly preferred embodiments, kits comprising (a) a basecoat composition, preferably comprising at least one pH or oxidation activating agent; and (b) a topcoat composition comprising at least one stimulus sensitive colorant, preferably an encapsulated pH or oxidation sensitive colorant, are provided. Such kits may also include other compositions or components such as, for example, a cosmetic removing composition, instructions for applying or using the compositions in the kit, cosmetic application devices (for example, a mascara brush, an eylash curling tool or application pad) which may or may not be capable of being heated, etc.

The packaging and application device for any subject of the invention may be chosen and manufactured by persons skilled in the art on the basis of their general knowledge, and adapted according to the nature of the composition to be packaged. Indeed, the type of device to be used can be in particular linked to the consistency of the composition, in particular to its viscosity; it can also depend on the nature of the constituents present in the composition, such as the presence of volatile compounds.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements.

### Example 1

### Temperature Sensitive Composition

Temperature Sensitive Mascara

| PHASE | INCI NAME | % |
|---|---|---|
| A1 | Stearic Acid | 7.40 |
| | Beeswax | 4.10 |
| | COPERNICIA CERIFERA (CARNAUBA) | 3.50 |
| | WAX | |
| | Paraffin | 12.60 |
| B1 | Distilled Water | Q.S. for 100% |
| | Preservative | 1.80 |
| B2 | Cellulose Gum | 1.38 |
| B4 | Triethanolamine | 2.40 |
| C | film former | 10.00 |
| D | Temperature sensitive colorant | 2 to 10% |
| | Total | 100% |

The composition was initially red. After heating, the composition turned white. After cooling in the refrigerator, the composition turned red.

### Example 2

### Pressure Sensitive Composition

5 grams of pressure sensitive colorant (Encapsulated Leuco Dyes) was added into 5 grams of latex film former (Daitosol 5000 SJ). The composition was then applied to paper, and pressure was applied. The composition turned blue after application of pressure.

### Example 3

### pH or Oxidation Sensitive Composition

A 2% salicylic acid in ethanol composition was applied over eyelashes coated with a pH sensitive Leuco dye. After application of the salicylic acid solution, the Leuco dye changed color.

What is claimed is:

1. A composition comprising a cosmetically acceptable medium and at least one stimulus sensitive colorant.

2. The composition according to claim 1, further comprising at least one non stimulus sensitive colorant.

3. The composition according to claim 1, further comprising water.

4. The composition according to claim 1, further comprising at least one oil.

5. The composition according to claim 1, further comprising at least one film-forming agent.

6. The composition according to claim 1, wherein the composition is a mascara.

7. The composition according to claim 3, wherein the composition is an emulsion.

8. The composition according to claim 4, wherein the composition is anhydrous.

9. A method of enhancing the appearance of eyelashes, fingernails, lips or skin comprising applying a composition comprising at least one stimulus sensitive colorant to the skin, eyelashes, lips or fingernails, and applying a stimulus to the composition to effect color change of the composition.

10. The method of claim 9, wherein the composition is applied to eyelashes.

11. The method of claim 9, wherein the composition is applied to skin.

12. The method of claim 9, wherein the composition is applied to lips.

13. The method of claim 9, wherein the composition is applied to fingernails.

14. The method of claim 9, wherein applying a stimulus to the composition comprises applying pressure to the composition.

15. The method of claim 9, wherein applying a stimulus to the composition comprises heating the composition.

16. The method of claim 9, wherein applying a stimulus to the composition comprises cooling the composition.

17. The method of claim 9, wherein applying a stimulus to the composition comprises applying a second composition comprising at least one pH or oxidation activating agent to the composition comprising at least one stimulus sensitive colorant.

18. A kit comprising (a) a composition comprising at least one stimulus sensitive colorant; and (b) means for providing a stimulus sufficient to effect color change of the composition.

19. The kit according to claim 18, wherein the means for providing a stimulus sufficient to effect color change of the composition is an eyelash curling tool or a mascara brush.

20. The kit according to claim 19, wherein the eyelash curling tool or mascara brush comprises a heating element.
